(19) United States
(12) Patent Application Publication
(10) Pub. No.: US 2024/0200676 A1
(43) Pub. Date: Jun. 20, 2024

(54) CONICAL CHECK VALVES

(71) Applicant: CareFusion 303, Inc., San Diego, CA (US)

(72) Inventor: Raymond P. Feith, Chino Hills, CA (US)

(21) Appl. No.: 18/536,647

(22) Filed: Dec. 12, 2023

Related U.S. Application Data
(60) Provisional application No. 63/432,667, filed on Dec. 14, 2022.

(51) Int. Cl. F16K 15/14

(52) U.S. Cl. CPC F16K 15/145

(57) ABSTRACT

A check valve includes an upper housing defining an inlet of the check valve, a lower housing defining an outlet of the check valve, and a cavity interposed between and defined by the upper and lower housings for fluidly connecting the inlet and the outlet. The check valve further includes a valve member mounted in the cavity to selectively permit fluid flow in a first direction, and prevent fluid backflow in a second direction opposite to the first direction. The valve member includes a valve body and a valve stem portion extending axially through a central axis of the valve body. The valve member may further include a plurality of feet disposed about and extending longitudinally from an outer circumferential perimeter of the valve body.

### CROSS REFERENCE TO RELATED APPLICATION

This application claims the benefit of priority under 35 U.S.C. § 119 to U.S. Provisional Patent Application Ser. No. 63/432,667, entitled “CONICAL CHECK VALVES”, filed on Dec. 14, 2022, the entire contents of which is incorporated herein by reference.

### TECHNICAL FIELD

The present disclosure generally relates to check valves, and more particularly to conical check valves having geometries capable of filtering particulate and minimizing backcheck leaks in the check valve.

### BACKGROUND

Patients are commonly injected with IV solutions that are initially provided in an IV reservoir (a bottle or bag) and dripped into the vein of the patient through an IV line. Typically, an injection port is provided along the IV line and adapted to function with a syringe to permit an injectate to be added to the IV solution. A check valve is also commonly included in the IV line to permit fluid flow only in the direction of the patient. This ensures that the injectate flows downstream toward the patient, not upstream toward the IV reservoir. Conventional check valves utilize disc-shaped valve members that are generally flat and do not have filters inherent to the geometry.

### SUMMARY

The present disclosure generally relates to check valves, and more particularly to valve members of check valves having geometries capable of filtering particulate and minimizing backflow as the fluid flows through the valve.

In accordance with various embodiments of the present disclosure, a check valve includes an upper housing, a lower housing, a cavity interposed between and defined by the upper and lower housings, and a valve member mounted in the cavity to selectively permit fluid flow in a first direction and prevent fluid backflow in a second direction opposite to the first direction. The upper housing defines an inlet of the check valve and the lower housing defines an outlet of the check valve. The cavity fluidly connects the inlet and the outlet. The valve member includes a valve body and a valve stem portion extending axially through a central axis of the valve body.

In accordance with various embodiments of the present disclosure, a check valve includes an upper housing defining an inlet of the check valve, a lower housing axially coupled to the upper housing and comprising an outlet of the check valve, and a cavity interposed between and defined by the upper and lower housings for fluidly connecting the inlet and the outlet. The check valve further includes a flexible valve member mounted in the cavity to selectively permit fluid flow in a first direction and prevent fluid backflow in a second direction opposite to the first direction.

Embodiments of the present disclosure provide a check valve, comprising an upper housing defining an inlet of the check valve and including a core member, a lower housing defining an outlet of the check valve, wherein a cavity is interposed between and defined by the upper housing and the lower housing for fluidly connecting the inlet and the outlet and a flexible valve member mounted in the cavity to selectively permit fluid flow in a first direction and prevent fluid backflow in a second direction opposite to the first direction.

In some embodiments, the check valve further comprises a plurality of filtering grooves on an outer perimeter of the core of the upper housing. In some embodiments, plurality of filtering grooves each define a recessed flow portion through which fluid entering the cavity flows from the upper housing into the lower housing. In some embodiments, the flexible valve member is stretched around the core member of the upper housing to shut off against the filtering grooves. In some embodiments, the flexible valve member is made of silicon. In some embodiments, a sealing surface is defined at a distal end of the core member of the upper housing, and in a closed state, the flexible valve member is configured to contact the sealing surface to limit fluid flow past the scaling surface.

In some embodiments, when an upstream pressure is applied to the flexible valve member, the flexible valve member is configured to deflect away from the sealing surface to fluidly communicate the inlet and the cavity and when a downstream pressure is applied to the flexible valve member, the flexible valve member is configured to deflect towards the sealing surface to block the fluid communication between the inlet and the cavity and restrict backflow of the fluid from the outlet to the inlet.

Embodiments of the present disclosure provide a check valve, comprising an upper housing defining an inlet of the check valve and including a core member, a lower housing axially coupled to the upper housing and comprising an outlet of the check valve, wherein a cavity is formed between and defined by the upper and lower housings for fluidly connecting the inlet and the outlet, a flexible valve member mounted in the cavity to selectively permit fluid flow in a first direction and prevent fluid backflow in a second direction opposite to the first direction, the upper housing having a plurality of filtering grooves disposed about an outer circumferential perimeter of the core member of the upper housing and a sealing surface defined at a distal end of the core member of the upper housing.

In some embodiments, the flexible valve member is configured to deflect away from the sealing surface in a high flow state, allowing the check valve to be flushed. In some embodiments, when a downstream pressure is applied to the flexible valve member, the flexible valve member is configured to deflect towards the sealing surface to block fluid communication between the inlet and the cavity and restrict backflow of the fluid from the outlet to the inlet.

Embodiments of the present disclosure include a method of providing a check valve, comprising providing an upper housing defining an inlet of the check valve and including a core member, providing a lower housing axially coupled to the upper housing and comprising an outlet of the check valve, providing a cavity interposed between and defined by the upper and lower housings for fluidly connecting the inlet and the outlet and providing a flexible valve member mounted in the cavity to selectively permit fluid flow in a first direction and prevent fluid backflow in a second direction opposite to the first direction, the upper housing having a plurality of filtering grooves disposed about an outer circumferential perimeter of the core member of the upper housing.

In some embodiments, the flexible valve member is configured to deflect away from the sealing surface in a high flow state, allowing the check valve to be flushed. In some embodiments, when a downstream pressure is applied to the flexible valve member, the flexible valve member is configured to deflect towards the sealing surface to block fluid communication between the inlet and the cavity and restrict backflow of the fluid from the outlet to the inlet.

In accordance with various embodiments of the present disclosure, the upper housing has a core, and the core has a plurality of filtering grooves extending longitudinally from an outer circumferential perimeter of the core of the upper housing.

It is to be understood that both the foregoing general description and the following detailed description are exemplary and explanatory and are intended to provide further explanation of the subject technology as claimed. It is also to be understood that other aspects may be utilized, and changes may be made without departing from the scope of the subject technology

### BRIEF DESCRIPTION OF THE DRAWINGS

The following figures are included to illustrate certain aspects of the embodiments and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a perspective cross-sectional view of a check valve, in accordance with some embodiments of the present disclosure.

FIG. 2 is an exploded perspective view of the check valve of FIG. 1, in accordance with some embodiments of the present disclosure.

FIGS. 3A-3B are cross-sectional views of the check valve of FIG. 1, in accordance with some embodiments of the present disclosure.

FIG. 4 is a cross-sectional view of the check valve of FIG. 1 in the closed state, wherein the check valve restricts fluid flow in the reverse directions, in accordance with some embodiments of the present disclosure.

FIGS. 5A-5B are cross-sectional views of the check valve of FIG. 1 in the closed state, illustrating the fluid flow through the check valve in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

The present description relates in general to check valves, and more particularly, for example and without limitation, to more particularly to conical check valves having the capability of filtering particulate and minimizing backcheck leaks in the check valve.

In some embodiments, the check valve is used in an IV administration set having a structure for coupling with an IV bag and/or a drip chamber. The structure for the IV bag can be formed as an arm, hook, clamp, or another mechanism configured to suspend the IV bag.

In accordance with some embodiments, the check valve may be a three-piece assembly, including an upper housing of the check valve (where the inlet is located), a lower housing of the check valve (where the outlet is located), and a flexible valve member. The upper housing may include a plurality of filtering grooves on the outer perimeter of the core of the upper housing. In some embodiments, the flexible valve is made out of any rubber-like or resilient material (e.g., silicone). As shown, the flexible valve could be stretched over the core of the upper housing to shut off against the filtering grooves. The lower housing is then secured to the upper housing, with the flexible valve in between the upper and lower housings. The three-piece check valve assembly does not have to be radially aligned during assembly. For example, the upper housing, the lower housing, and flexible valve can be aligned in any radial orientation and the desired results are achieved.

The lower housing may be coupled, attached or otherwise bonded to a ledge of the internal surface of the upper housing through any appropriate methods including, but not limited to ultrasonic welding, heat sealing, insert molding, gluing or other attachment methods. The check valve can be flushed with high fluid flow, pushing the flexible valve outwards away from the filtering grooves and allowing fluid to flow around the entire perimeter of the core of upper housing.

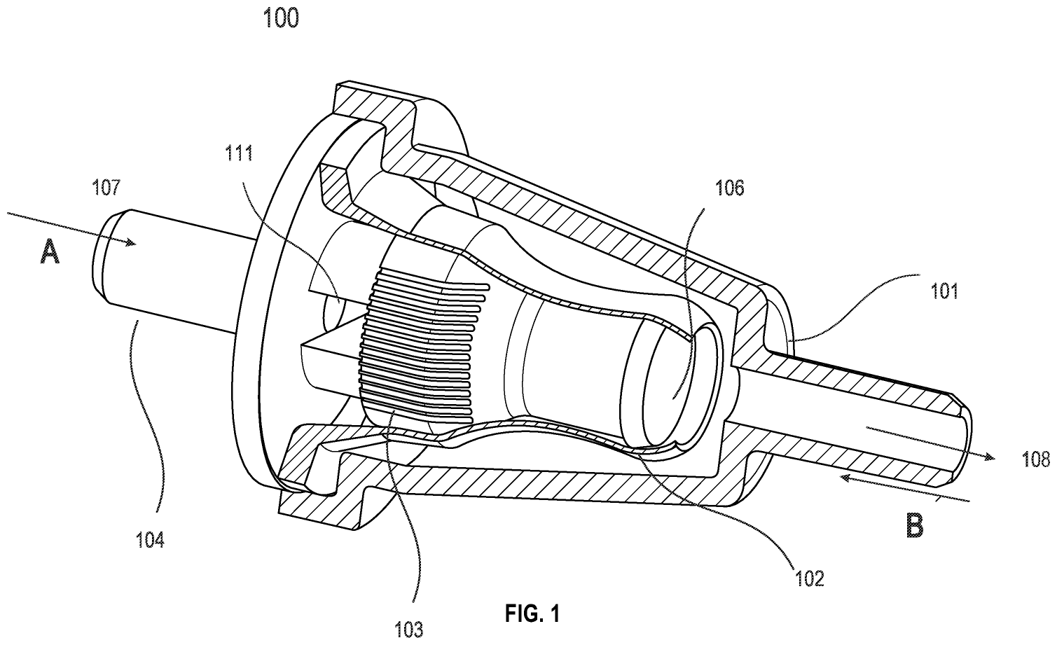

FIG. 1 is a perspective view of a check valve 100, in accordance with some embodiments of the present disclosure. As depicted, a portion of the check valve 100 (a lower housing 101 and a flexible valve 102) is displayed in cross-sectional view to illustrate some of the features of the filtering grooves 103 and the flexible valve 102. FIG. 1 shows a check valve 100 in a closed state, including an upper housing 104, a lower housing 101, and a flexible valve member 102 installed between the upper housing 104 and the lower housing 101.

The three-piece check valve assembly 100 as shown in FIG. 1 does not have to be radially oriented during manufacturing assembly. In other words, the upper housing 104, the flexible valve member 102, and the lower housing 101 could be assembled with the filtering grooves in any radial position. The aforementioned configuration of the check valve assembly 100 provides manufacturing and assembly advantages.

The plurality filtering grooves 103 may be configured to restrict and minimize passage of undesirable matter in the fluid flowing through the check valve 100. If not filtered, the undesirable matter could otherwise cause damage or wear to the check valve. The aforementioned configuration also prevents the undesirable particulate matter from potentially becoming lodged between the lower portion of the core of the inlet 107 and the sealing surface 106, thereby preventing the flexible valve member 102 from fully closing and sealing against reverse flow (backflow).

During operation, when a downstream pressure (i.e., a pressure applied by a fluid flowing from the outlet 108 to the inlet 107) is applied to the flexible valve member 102, the flexible valve member 102 may deflect towards the sealing surface 106 on the upper housing 110 to block the fluid communication between the inlet 107 and the cavity 109, thereby restricting backflow of the fluid from the outlet 108 to the inlet 107. Preventing backflow of the fluid is advantageous in that it restricts undesirable particulate matter, for example, contained in a drug dispensed from a secondary path from flowing back through the check valve 100, thereby preventing the patient from receiving the proper drug dosage concentration or from timely delivery of the drug.

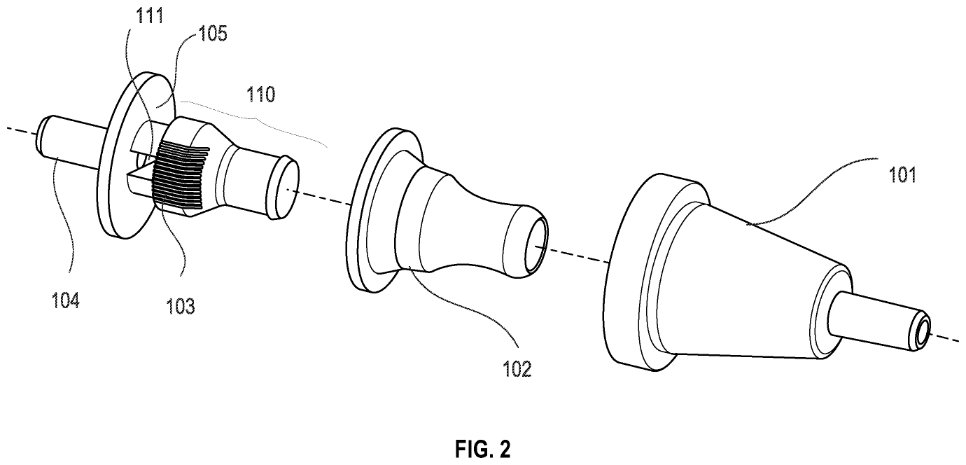

As depicted, during operation, fluid may enter the check valve 100 via the inlet 107, and flow through the filtering grooves 103 where it is filtered to trap the undesirable particulate matter, and into the cavity 109. As illustrated in FIGS. 1 and 2, a cavity 111 defined by two semi-cylindrical components in an upper portion of the core of the upper housing 110, right next to the inlet 107, may assist in directing the fluid flow towards the filtering grooves 103 once the fluid enters the inlet 107. Any grit or other undesirable particulate matter larger in size than the filtering grooves 103 may be trapped in the filtering grooves 103 and prevented from passing downstream to the seal ring. The upstream pressure (i.e., pressure applied by fluid flowing from the inlet 107 to the outlet 108) applied to the flexible valve member 102 causes the flexible valve member 102 to bow or bend outwards and deflect away from the sealing surface 106. Thus, the flexible valve member 102 is shifted from the closed state to an open state where the inlet 107, the cavity 109, and the outlet 108 are fluidly communicated. In the open state, a gap may be created between the sealing surface 106 and the flexible valve to allow the filtered fluid to flow therethrough. The filtered fluid may then flow through the gap and exit the check valve 100 via the outlet 108 in the lower housing 101.

In contrast, in a conventional check valve configuration which does not include an integrated filter, during low flow conditions, pressure exerted on the check valve as a result of the fluid flow may not be sufficient to fully open the check valve (e.g., to deflect the flexible valve member 102) such that grit (or other undesirable particulate matter) may pass through the gap. In such conditions, the grit may get lodged in the gap and the valve may not completely close. This undesirably causes the check valve to “weep,” and allow fluid to flow through the valve in the reverse direction, thereby making the check valve ineffective.

In some embodiments, the upper housing 104, flexible valve member 102, and the lower housing 101 are not limited to any particular shape or size. In the depicted embodiments, however, the size of the upper housing 104, flexible valve member 102, and the lower housing 101 may be limited based on desired deflection/bending characteristics of the upper housing 104, flexible valve member 102, and the lower housing 101 when subjected to either of the upstream or downstream forces. For example, the upper housing 104, flexible valve member 102, and the lower housing 101 may be sized and shaped so as to flex or bend under fluid pressure to permit forward flow (from the inlet 107 to the outlet 108) of the fluid into the cavity 109, and to limit fluid flow in the reverse direction.

FIG. 2 is an exploded view of the three-piece check valve assembly 100. The flexible valve 102 extends around, and may be stretched around, the core of the upper housing 110 to close off the filtering grooves 103. The flange of the flexible valve member 102 may then be coupled to the ledge of the internal surface 105 of the upper housing 110 through any appropriate methods including, but not limited to ultrasonic welding, heat sealing, insert molding, gluing or other attachment methods to create a hermetic seal.

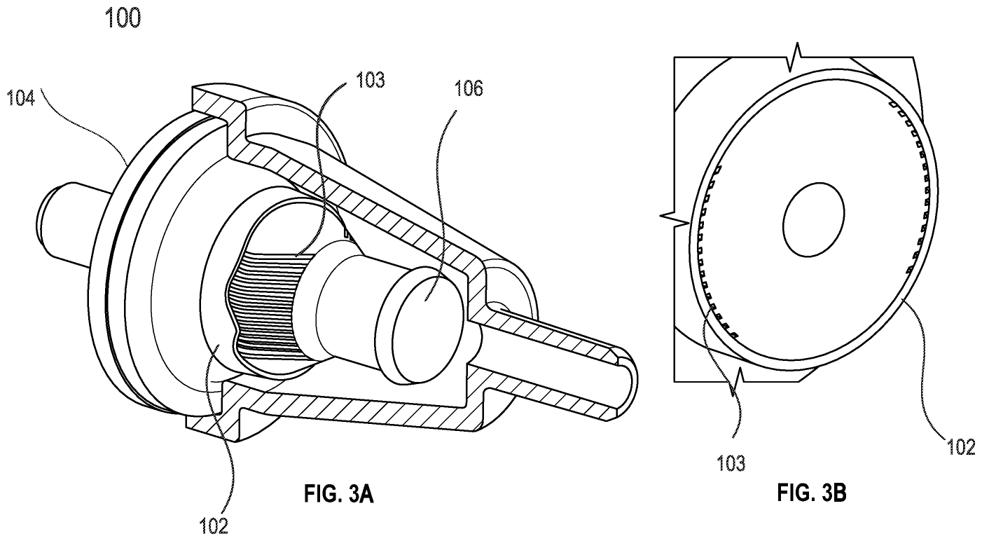

FIGS. 3A-3B are cross-sectional views of the check valve 100, specifically illustrating the filtering grooves 103. The flexible valve 102 extends around the core of the upper housing 110 to close off the filtering grooves 103. As shown in FIG. 3B, the fluid is able to flow through filtering grooves 103 when the flexible valve 102 is stretched around the core of the upper housing 110 in a low flow state. In some embodiments, the fluid is only able to flow through the filtering grooves 103 in a low flow state.

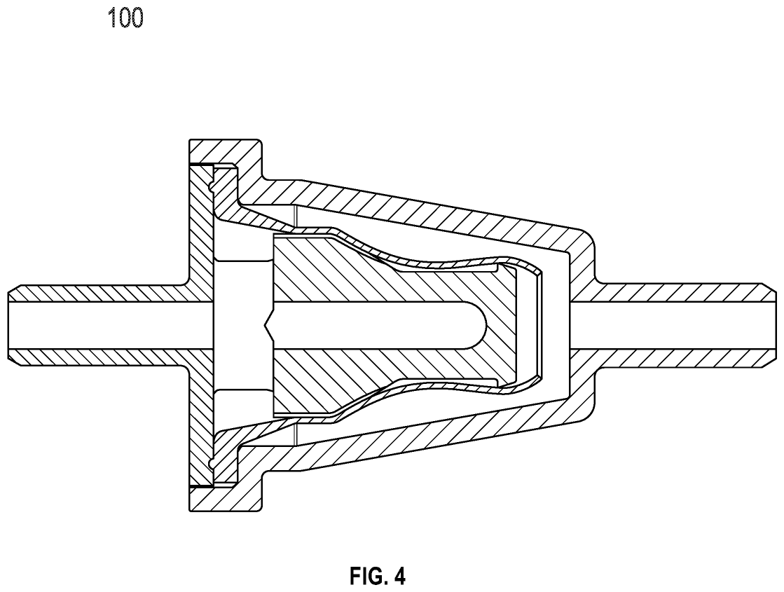

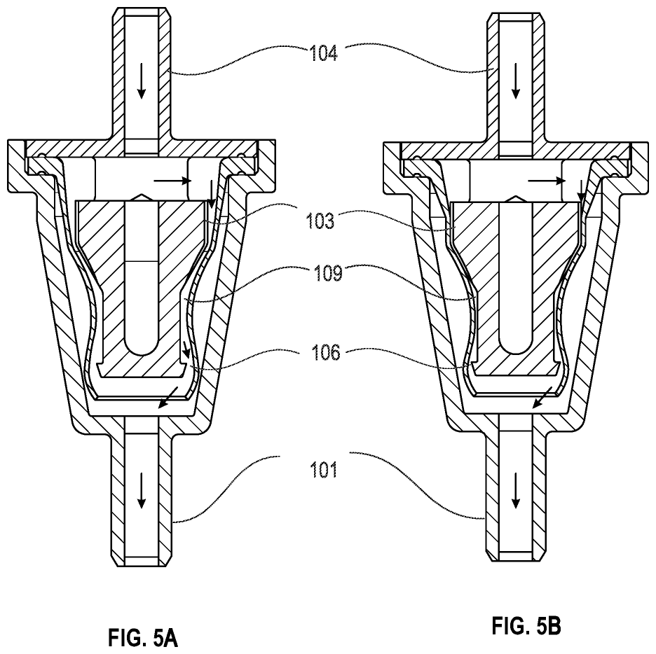

FIGS. 4 and 5A-5B are cross-sectional views of the three-piece check valve assembly. The arrows in FIGS. 5A-5B show the path of fluid flow starting at the inlet 107, through filtering grooves 103, around the sealing surface 106, and through the outlet 108. FIG. 5A illustrates how the flexible valve 102 would react in a high fluid flow (flushing) situation and FIG. 5B shows how the flexible valve 102 would react in a low fluid flow state. In a high flow state, a flexible valve 102 may stretch and allow fluid to bypass the filtering grooves 103, which allows the check valve to be flushed of all debris.

In accordance with some embodiments, the flexible valve member 102 may be formed of a flexible, resilient material which is fluid impervious. For example, the flexible valve member 102 may be made of a silicon material. In other embodiments, however, the flexible valve member 102 may be formed of any non-sticking, resilient material such as biocompatible natural or synthetic rubber or plastic.

The upper housing 104 may include an inlet 107 of the check valve 100 at the first end, and the lower housing 101 may include an outlet 108 of the check valve 100. The check valve may define an internal flow cavity 109 axially extending between the inlet 107 and the outlet 108 and in fluid communication therewith. As is understood, the check valve 100 may permit fluid to flow from the inlet 107 to the outlet 108 (as indicated by arrows in FIGS. 5A-5B and arrow A), and minimize, or otherwise limit, fluid flow from the outlet 108 to the inlet 107 (as indicated by arrow B). As depicted, the upper housing 104, the flexible valve member 102 lower housing 101 may define the cavity 109 for fluidly connecting the inlet 107 and the outlet 108. In the depicted embodiments, the flexible valve member 102 may be stretched around the core of the upper housing 110 to selectively permit fluid flow in the first direction (indicated by arrow A) and prevent fluid backflow (reverse flow) in the second direction opposite to the first direction (indicated by arrow B).

Various examples of aspects of the disclosure are described as numbered clauses (1, 2, 3, etc.) for convenience. These are provided as examples and do not limit the subject technology. Identification of the figures and reference numbers are provided below merely as examples for illustrative purposes, and the clauses are not limited by those identifications.

Clause 1: A check valve, comprising an upper housing defining an inlet of the check valve and including a core member, a lower housing defining an outlet of the check valve, wherein a cavity is interposed between and defined by the upper housing and the lower housing for fluidly connecting the inlet and the outlet, and a flexible valve member mounted in the cavity to selectively permit fluid flow in a first direction and prevent fluid backflow in a second direction opposite to the first direction.

Clause 2: The check valve of clause 1, further comprising a plurality of filtering grooves on an outer perimeter of the core of the upper housing.

Clause 3: The check valve of clause 2, wherein plurality of filtering grooves each define a recessed flow portion through which fluid entering the cavity flows from the upper housing into the lower housing.

Clause 4: The check valve of clause 3, wherein the flexible valve member is stretched around the core member of the upper housing to shut off against the filtering grooves.

Clause 5: The check valve of clause 1, wherein the flexible valve member is made of silicon.

Clause 6: The check valve of clause 1, wherein a sealing surface is defined at a distal end of the core member of the upper housing and in a closed state,

Clause 7: The check valve of clause 1, wherein the flexible valve member is configured to contact the sealing surface to limit fluid flow past the sealing surface.

Clause 8: The check valve of clause 6, wherein when an upstream pressure is applied to the flexible valve member, the flexible valve member is configured to deflect away from the sealing surface to fluidly communicate the inlet and the cavity.

Clause 9: The check valve of clause 6, wherein when a downstream pressure is applied to the flexible valve member, the flexible valve member is configured to deflect towards the sealing surface to block the fluid communication between the inlet and the cavity and restrict backflow of the fluid from the outlet to the inlet.

Clause 10: The check valve of clause 2, wherein the plurality of filtering grooves does not extend around the entire perimeter of the core of the upper housing.

Clause 11: The check valve of clause 1, wherein the cavity is defined by two semi-cylindrical components in an upper portion of the core member.

Clause 12: The check valve of clause 1, wherein the upper housing, the flexible valve member, and the lower housing is sized and shaped so as to flex or bend under fluid pressure to permit forward flow of the fluid into the cavity, and to limit fluid flow in the reverse direction.

Clause 13: The check valve of clause 2, wherein grit or other undesirable particulate matter larger in size than the filtering grooves is trapped in the filtering grooves and prevented from passing downstream.

Clause 14: A check valve, comprising an upper housing defining an inlet of the check valve and including a core member, a lower housing axially coupled to the upper housing and comprising an outlet of the check valve, wherein a cavity is formed between and defined by the upper and lower housings for fluidly connecting the inlet and the outlet, a flexible valve member mounted in the cavity to selectively permit fluid flow in a first direction and prevent fluid backflow in a second direction opposite to the first direction, the upper housing having a plurality of filtering grooves disposed about an outer circumferential perimeter of the core member of the upper housing, and a sealing surface defined at a distal end of the core member of the upper housing.

Clause 15: The check valve of clause 14, wherein the flexible valve member is configured to deflect away from the sealing surface in a high flow state, allowing the check valve to be flushed.

Clause 16: The check valve of clause 14, wherein when an upstream pressure is applied to the flexible valve member, the flexible valve member is configured to deflect away from the sealing surface to fluidly communicate the inlet and the cavity, and when a downstream pressure is applied to the flexible valve member, the flexible valve member is configured to deflect towards the sealing surface to block fluid communication between the inlet and the cavity and restrict backflow of the fluid from the outlet to the inlet.

Clause 17: A method of providing a check valve, comprising providing an upper housing defining an inlet of the check valve and including a core member, providing a lower housing axially coupled to the upper housing and comprising an outlet of the check valve, providing a cavity interposed between and defined by the upper and lower housings for fluidly connecting the inlet and the outlet; and providing a flexible valve member mounted in the cavity to selectively permit fluid flow in a first direction and prevent fluid backflow in a second direction opposite to the first direction, the upper housing having a plurality of filtering grooves disposed about an outer circumferential perimeter of the core member of the upper housing.

Clause 18: The method of clause 17, wherein the flexible valve member is configured to deflect away from the sealing surface in a high flow state, allowing the check valve to be flushed.

Clause 19: The method of clause 17, wherein when an upstream pressure is applied to the flexible valve member, the flexible valve member is configured to deflect away from the sealing surface to fluidly communicate the inlet and the cavity.

Clause 20: The method of clause 17, wherein when a downstream pressure is applied to the flexible valve member, the flexible valve member is configured to deflect towards the sealing surface to block fluid communication between the inlet and the cavity and restrict backflow of the fluid from the outlet to the inlet.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A check valve, comprising:
an upper housing defining an inlet of the check valve and including a core member;
a lower housing defining an outlet of the check valve, wherein a cavity is interposed between and defined by the upper housing and the lower housing for fluidly connecting the inlet and the outlet; and
a flexible valve member mounted in the cavity to selectively permit fluid flow in a first direction and prevent fluid backflow in a second direction opposite to the first direction.

2. The check valve of claim 1, further comprising a plurality of filtering grooves on an outer perimeter of the core of the upper housing.

3. The check valve of claim 2, wherein plurality of filtering grooves each define a recessed flow portion through which fluid entering the cavity flows from the upper housing into the lower housing.

4. The check valve of claim 3, wherein:
the flexible valve member is stretched around the core member of the upper housing to shut off against the filtering grooves.

5. The check valve of claim 1, wherein the flexible valve member is made of silicon.

6. The check valve of claim 1, wherein a sealing surface is defined at a distal end of the core member of the upper housing.

7. The check valve of claim 1, wherein in a closed state, the flexible valve member is configured to contact the sealing surface to limit fluid flow past the sealing surface.

8. The check valve of claim 6, wherein when an upstream pressure is applied to the flexible valve member, the flexible valve member is configured to deflect away from the sealing surface to fluidly communicate the inlet and the cavity.

9. The check valve of claim 6, wherein when a downstream pressure is applied to the flexible valve member, the flexible valve member is configured to deflect towards the sealing surface to block the fluid communication between the inlet and the cavity and restrict backflow of the fluid from the outlet to the inlet.

10. The check valve of claim 2, wherein the plurality of filtering grooves does not extend around the entire perimeter of the core of the upper housing.

11. The check valve of claim 1, wherein the cavity is defined by two semi-cylindrical components in an upper portion of the core member.

12. The check valve of claim 1, wherein the upper housing, the flexible valve member, and the lower housing is sized and shaped so as to flex or bend under fluid pressure to permit forward flow of the fluid into the cavity, and to limit fluid flow in the reverse direction.

13. The check valve of claim 2, wherein grit or other undesirable particulate matter larger in size than the filtering grooves is trapped in the filtering grooves and prevented from passing downstream.

14. A check valve, comprising:
an upper housing defining an inlet of the check valve and including a core member;
a lower housing axially coupled to the upper housing and comprising an outlet of the check valve, wherein a cavity is formed between and defined by the upper and lower housings for fluidly connecting the inlet and the outlet;
a flexible valve member mounted in the cavity to selectively permit fluid flow in a first direction and prevent fluid backflow in a second direction opposite to the first direction, the upper housing having a plurality of filtering grooves disposed about an outer circumferential perimeter of the core member of the upper housing; and
a sealing surface defined at a distal end of the core member of the upper housing.

15. The check valve of claim 14, wherein the flexible valve member is configured to deflect away from the sealing surface in a high flow state, allowing the check valve to be flushed.

16. The check valve of claim 14, wherein:
when an upstream pressure is applied to the flexible valve member, the flexible valve member is configured to deflect away from the sealing surface to fluidly communicate the inlet and the cavity, and
when a downstream pressure is applied to the flexible valve member, the flexible valve member is configured to deflect towards the sealing surface to block fluid communication between the inlet and the cavity and restrict backflow of the fluid from the outlet to the inlet.

17. A method of providing a check valve, comprising:
providing an upper housing defining an inlet of the check valve and including a core member;
providing a lower housing axially coupled to the upper housing and comprising an outlet of the check valve;
providing a cavity interposed between and defined by the upper and lower housings for fluidly connecting the inlet and the outlet; and
providing a flexible valve member mounted in the cavity to selectively permit fluid flow in a first direction and prevent fluid backflow in a second direction opposite to the first direction, the upper housing having a plurality of filtering grooves disposed about an outer circumferential perimeter of the core member of the upper housing.

18. The method of claim 17, wherein the flexible valve member is configured to deflect away from the sealing surface in a high flow state, allowing the check valve to be flushed.

19. The method of claim 17, wherein when an upstream pressure is applied to the flexible valve member, the flexible valve member is configured to deflect away from the sealing surface to fluidly communicate the inlet and the cavity.

20. The method of claim 17, wherein when a downstream pressure is applied to the flexible valve member, the flexible valve member is configured to deflect towards the sealing surface to block fluid communication between the inlet and the cavity and restrict backflow of the fluid from the outlet to the inlet.
